(19) United States
(12) Patent Application Publication
(10) Pub. No.: US 2019/0184853 A1
(43) Pub. Date: Jun. 20, 2019

(54) OCCUPANT SENSING SYSTEM FOR A VEHICLE

(71) Applicant: GM Global Technology Operations LLC, Detroit, MI (US)

(72) Inventors: Scott D. Thomas, Novi, MI (US); Dorel M. Sala, Troy, MI (US); Tricia E. Morrow, Grosse Pointe, MI (US); Chad M. O'Brien, Shelby Twp., MI (US)

(21) Appl. No.: 15/847,044

(22) Filed: Dec. 19, 2017

(51) Int. Cl. B60N 2/00; B60R 21/015; B60R 22/48; A61B 5/00

(52) U.S. Cl. CPC B60N 2/002; B60R 21/01516; B60R 21/01566; B60R 21/01552; B60R 21/01556; A61B 5/04085; A61B 5/6893; B60R 2022/4858; B60R 2022/4816; B60R 2022/4866; B60R 22/48

(57) ABSTRACT

An occupant detection system includes a vehicle seat having a seat back and a seat base, and a biometric sensor arranged in at least one of the seat back and the seat base. The biometric sensor is configured to detect a physiological characteristic of a seat occupant. A controller is operatively connected to the biometric sensor. The controller is configured to provide an output signal in response to a physiological characteristic detected signal from the biometric sensor.

### INTRODUCTION

The subject disclosure relates to the art of vehicles and, more particularly, to an occupant sensing system for a vehicle.

Many current vehicles include sensors for detecting a presence in a vehicle seat. Sensors may take the form of pressure sensors and detect the presence based on weight. Typically, the sensors are connected to a controller that is configured to provide an output signal when a mass having a weight exceeding a predetermined threshold is detected in the seat. The output may be employed to generate a passenger primary restraint warning signal (seat belt), generate signals to control a supplemental restraint system (airbags/seat belt pretensioners) for normally seated adult occupants, or enable entertainment system functionality.

The predetermined threshold is set to capture people of a certain size and exclude other seat occupants such as pets and packages. The predetermined threshold often times captures children in child safety seats. In many cases, child safety seats do not utilize existing passenger restraints but rather rely on dedicated attachment systems. Thus, often times, a passenger restraint warning may be issued unnecessarily. That is, a sensor may detect the presence of an occupied child safety seat that does not utilize the passenger restraint system. Accordingly, it is desirable to provide a system that can differentiate between an occupant sitting directly on a vehicle seat and an occupant that may occupy a child safety seat that does not use the primary passenger restraint system.

### SUMMARY

Disclosed is an occupant detection system including a vehicle seat having a seat back and a seat base, and a biometric sensor arranged in at least one of the seat back and the seat base. The biometric sensor is configured to detect a physiological characteristic of a seat occupant. A controller is operatively connected to the biometric sensor. The controller is configured to provide an output signal in response to a physiological characteristic detected signal from the biometric sensor.

In addition to one or more of the features described herein include wherein the biometric sensor comprises at least one of an electrocardiogram (EKG) sensor, an electroencephalogram (EEG) sensor, an electromyography (EMG) sensor, a piezo electric sensor, a neural activity sensor, and a heart activity sensor.

In addition to one or more of the features described herein include wherein the biometric sensor is arranged in the seat base.

In addition to one or more of the features described herein include wherein the biometric sensor is arranged in the seat back.

In addition to one or more of the features described herein include wherein the biometric sensor is arranged between the seat back and the seat base.

In addition to one or more of the features described herein include wherein the biometric sensor establishes a physiological characteristic detection zone at the vehicle seat.

In addition to one or more of the features described herein include a non-biometric sensor configured to detect a mass of a seat occupant.

Also disclosed is a vehicle including a body defining, at least in part, an occupant compartment. At least one vehicle seat is arranged in the occupant compartment. The at least one vehicle seat includes a seat back and a seat base. A biometric sensor is arranged in at least one of the seat back and the seat base. The biometric sensor is configured to detect a physiological characteristic of a seat occupant. A controller is operatively connected to the biometric sensor. The controller is configured to provide an output signal in response to a physiological characteristic detected signal from the biometric sensor.

In addition to one or more of the features described herein include wherein the biometric sensor comprises at least one of a an electrocardiogram (EKG) sensor, an electroencephalogram (EEG) sensor, an electromyography (EMG) sensor, a piezo electric sensor, a neural activity sensor, and a heart activity sensor.

In addition to one or more of the features described herein include wherein the biometric sensor is arranged in the seat base.

In addition to one or more of the features described herein include wherein the biometric sensor is arranged in the seat back.

In addition to one or more of the features described herein include wherein the biometric sensor is arranged between the seat back and the seat base.

In addition to one or more of the features described herein include wherein the biometric sensor establishes a physiological characteristic detection zone at the at least one vehicle seat.

In addition to one or more of the features described herein include a non-biometric sensor configured to detect a mass of a seat occupant.

Further disclosed is a method of detecting an occupant in a vehicle including sensing a physiological characteristic at a vehicle seat.

In addition to one or more of the features described herein include wherein sensing the physiological characteristic includes detecting at least one of an electrical activity of a brain, electrical activity of a heart, heart movement, and breathing activity.

In addition to one or more of the features described herein include detecting an occupant restraint status of the vehicle seat.

In addition to one or more of the features described herein include providing a signal based on a presence of the physiological characteristic and occupant restraint status.

In addition to one or more of the features described herein include wherein providing the signal includes at least one of limiting operation status of the vehicle and warning the occupant to buckle a seatbelt, controlling an airbag, a seatbelt pretensioner, an entertainment system, and an HVAC system.

In addition to one or more of the features described herein include detecting at least one of a child in a child safety seat resting on the vehicle seat, an occupant on the vehicle seat, an out-of-position occupant on the vehicle seat, an animal on the vehicle seat, and an inanimate object on the vehicle seat based on the sensed physiological characteristic.

The above features and advantages, and other features and advantages of the disclosure are readily apparent from the following detailed description when taken in connection with the accompanying drawings.

### BRIEF DESCRIPTION OF THE DRAWINGS

Other features, advantages and details appear, by way of example only, in the following detailed description, the detailed description referring to the drawings in which:

FIG. 1 depicts a vehicle including an occupant sensing system, in accordance with an aspect of an exemplary embodiment;

FIG. 2 depicts a vehicle seat including an occupant sensing system, in accordance with an aspect of an exemplary embodiment;

FIG. 3 depicts a block diagram illustrating the occupant sensing system, in accordance with an aspect of an exemplary embodiment; and

FIG. 4 depicts a flow chart illustrating a method of detecting vehicle occupants, in accordance with an aspect of an exemplary embodiment.

### DETAILED DESCRIPTION

The following description is merely exemplary in nature and is not intended to limit the present disclosure, its application or uses. It should be understood that throughout the drawings, corresponding reference numerals indicate like or corresponding parts and features. As used herein, the term module refers to processing circuitry that may include an application specific integrated circuit (ASIC), an electronic circuit, a processor (shared, dedicated, or group) and memory that executes one or more software or firmware programs, a combinational logic circuit, and/or other suitable components that provide the described functionality.

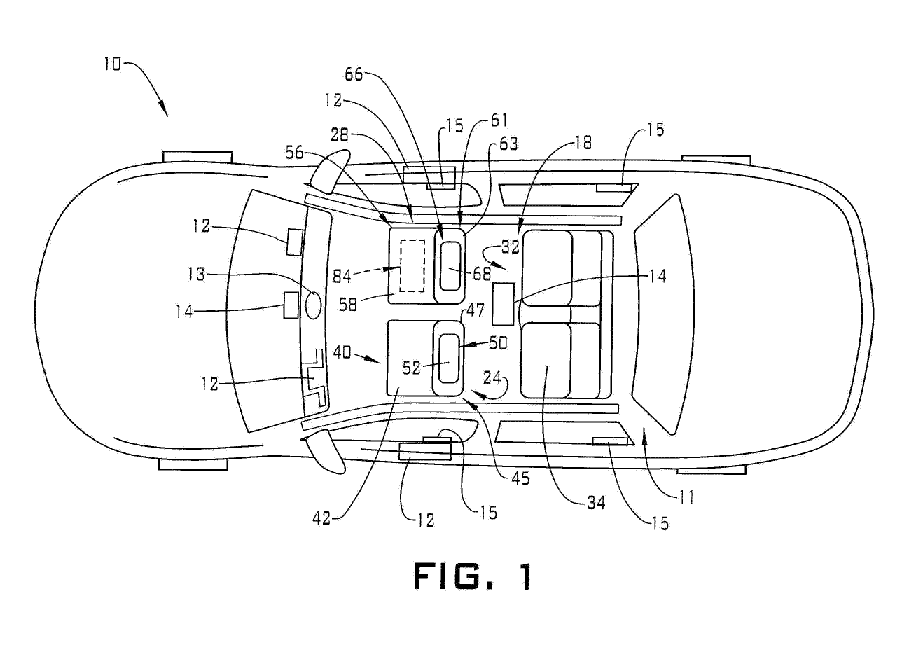

A vehicle, in accordance with an exemplary embodiment, is indicated generally at 10 in FIG. 1. Vehicle 10 includes a body 11 that defines, at least in part, an occupant compartment 18. Vehicle 10 may be powered by a variety of systems including internal combustion engines, hybrid electric engines, and electric motors (not shown). Vehicle 10 may be controlled by a driver, be remote controlled, or may be autonomous. Vehicle 10 may include one or more airbag systems 12. Airbag systems 12 may include side impact airbags, front impact airbags, rollover airbags, knee airbags and the like. Vehicle 10 may also include one or more entertainment systems 14 that can be instrument panel mounted, roof mounted seat mounted, center console mounted, door mounted and the like. In addition, vehicle 10 may include one or more heater/ventilation system HVAC climate controls 13 or other reachable controls 15.

A first vehicle seat 24 is arranged in occupant compartment 18. A second vehicle seat 28 is arranged alongside first vehicle seat 24, and a third vehicle seat 32 which may take the form of a bench seat 34 is arranged aft of first and second vehicle seats 24 and 28. At this point, it should be understood that the number, arrangement, and type of vehicle seats may vary. First vehicle seat 24 includes a first portion 40 which may define a seat base 42, a second portion 45 which may define a seat back 47 and a third portion 50 which may define a head restraint 52. Second vehicle seat 28 includes a first portion 56 which may define a seat base 58, a second portion 61 which may define a seat back 63 and a third portion 66 which may define a head restraint 68.

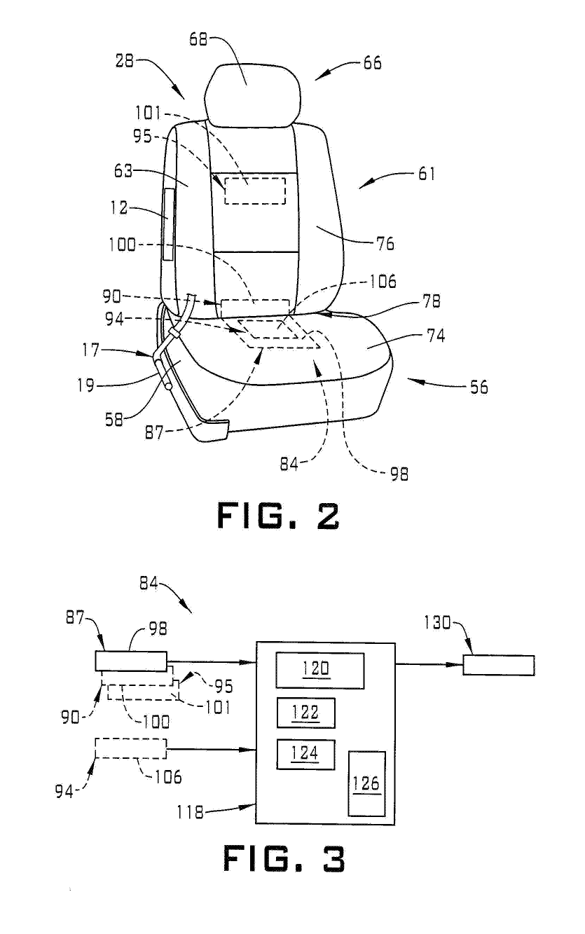

Reference will now follow to FIG. 2, with continued reference to FIG. 1, in describing second vehicle seat 28 with an understanding that first vehicle seat 24 and third vehicle seat 32 may include similar structure and seatbelt system. Seat base 58 includes a seat base surface 74, and seat back 63 includes a seat back surface 76. Second vehicle seat 28 also includes an interface zone 78 that is defined between seat base 58 and seat back 63. In addition second vehicle seat 28 may interface with a seatbelt system 17 that may include a pretensioner 19. Pretensioner 19 can be attached to an outboard lower anchor (not separately labeled), a seatbelt buckle (not shown), or a retractor (also not shown).

In accordance with an aspect of an exemplary embodiment, second vehicle seat 28 includes an occupant detection system 84. It should be understood that occupant detection system 84 may also be incorporated into first vehicle seat 24 and third vehicle seat 32, or any other designated seating position within any mode of personal transportation. Occupant detection system 84 includes a first sensor 87 that may be arranged in seat base 58 at seat base surface 74. A second sensor 90 may be arranged in seat back 63 at seat back surface 76. Additionally, a third sensor 94 may be arranged in seat base 58 and a fourth sensor 95 may be arranged in seat back 63 at a higher location than second sensor 90. It should be understood that first sensor 87 and/or second sensor 90 may extend into interface zone 78. Alternatively, a fifth sensor (not shown) may be arranged in interface zone 78. It should be understood that the number and location of sensors may vary.

First sensor 87 takes the form of a biometric sensor 98 that may detect a physiological characteristic of an occupant in second vehicle seat 28. If provided, second sensor 90 may also take the form of a biometric sensor 100. Further, fourth sensor 95 may also take the form of biometric sensor 101. The number and location of biometric sensors may vary and could include sensors at interface zone 78. Further, biometric sensors may take on various forms including, but not limited to, electrocardiogram (EKG) sensors, electroencephalogram (EEG) sensors such as a Freer neuro monitor based sensor, electromyography (EMG) sensors, and/or sensors that may detect other physiological characteristics including, but not limited to, piezo electric sensors that may detect micro motions associated with a heartbeat and ultra-high impedance heart activity sensors such as Plessy Epic sensors that may be configured to detect breathing, heart rate and the like. In addition, the biometric sensors may be used, in conjunction with non-biometric sensors, for detection of children left in vehicle.

Also, if provided, third sensor 94 may take the form of a non-biometric sensor 106 that can detect a mass in seat base 58. Non-biometric sensor 106 may take the form of a load sensor such as a pressure based bladder system, a capacitive sensor, and/or a force resistive sensor that may detect localized pressure at one or more zones on seat base 58.

Referring to FIG. 3, and with continued reference to FIG. 2, occupant detection system 84 includes a controller 118 that may include a central processing unit (CPU) 120, a memory module 122, a signal conditioning module 124 and an occupant detection module 126. Controller 118 is operatively connected to first sensor 87 and, if provided, second sensor 90 and third sensor 94. Signal conditioning module 124 conditions signals received through first, second and third sensors 87, 90 and 94.

A conditioned signal may be passed to occupant detection module 126 to determine if an occupant is present on seat base surface 74. Occupant detection module 126 may also obtain data from load sensor 106. An “occupant present” output could be based in part on whether a load is detected above a predetermined level that may be stored in memory module 122. If an “occupant present” determination is made, occupant detection system 84 may output, to an external control system (not shown), or use internally, a signal 130.

Occupant detection system 84 may send a another signal (not shown) indicating that an occupant is sitting slightly out of normal seating position, or occupant detection system 84 may output signal 130 when an occupant is sitting across two seating positions. Signal 130 may be used for other vehicle systems such as seat or vehicle comfort features, primary or secondary restraint systems deployment, enabling/disabling vehicle entertainment/convenience features in general. Signal 130 may also be used to report to the primary owner that a child or other object was left behind when the primary occupant exits the vehicle.

Occupant detection system 84 is configured to discriminate between an occupant on seat base surface 74 and, for example, a child in a child safety seat. Thus, occupant detection system 84 establishes a physiological characteristic detection zone or sensing zone that may extend no more than about four inches (10 centimeters) above seat base surface 74. It should be understood that the position of each sensor in a vehicle seat may vary in order to achieve one or more of a selected sensing zone, selected seat comfort and sensor durability. Sensors may be arranged between an outer covering and a foam layer, below the foam layer, or the like. Further, biometric sensor 98 and, if provided, biometric sensor 90, are arranged with a minimal lateral dimension so as to avoid detecting an appendage such as an arm or a leg that may dangle from a child safety seat.

In addition biometric sensor 100 may be positioned at a lower portion of seat back 63 in order to avoid detecting a back or bottom of a child in a backless booster child safety seat. In this manner, occupant detection system 84 can provide output signal 130 when an occupant is sitting in a vehicle seat and not when the seat is occupied by a child in a child safety seat that does not utilize a seat belt for restraint. As will be discussed herein, output signal 130 may be employed to accurately generate a seat belt warning signal providing a reminder to buckle up, control a supplemental restraint system (airbags 12, seatbelt pretensioners 19) based on occupant presence or when an out of position occupant is detected, affect other vehicle systems such as enabling entertainment system 14, HVAC system controls 13 and functionality of other control 15, or prevent operation of vehicle 10.

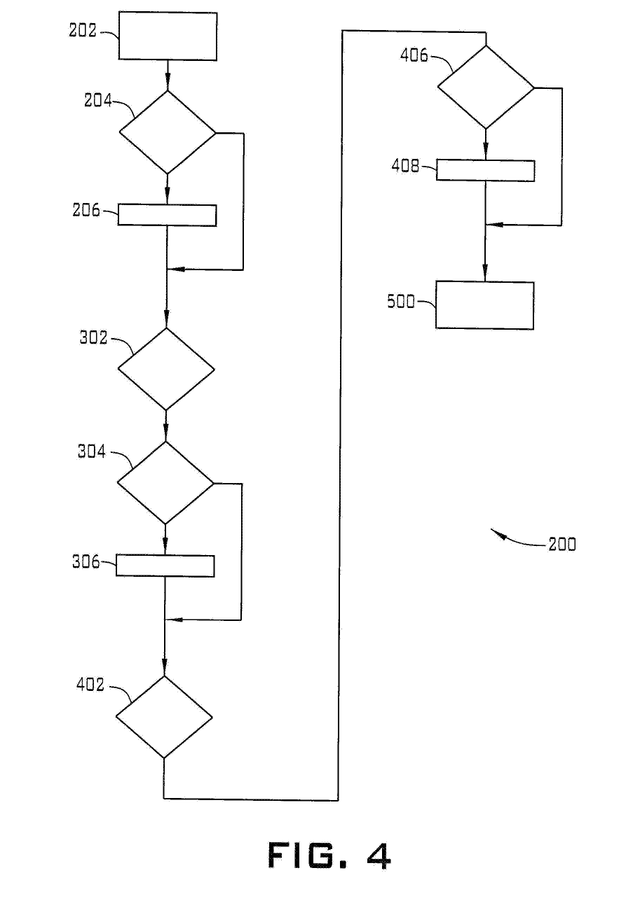

Reference will now follow to FIG. 4, with continued reference to FIGS. 1-3 in describing a method 200 of detecting occupants in a vehicle seat in accordance with an aspect of an exemplary embodiment. In block 202 first sensor 87, second sensor 90, and third sensor 94 are powered on by, for example, activating vehicle 10. Activating vehicle 10 may include, for example, opening a vehicle door (not separately labeled). In block 204 a determination is made whether physiological characteristics are sensed by first sensor 87 and, if provided, second sensor 90. Also, if provided, non-biometric sensor 106 may provide an input regarding a mass in, for example, second vehicle seat 28. If no occupant is detected, no action is taken. If an occupant is detected, the input is processed through filtering logic (not shown) and output signal 130 is issued and action may be taken in block 206. The action may include issuing a seat belt warning, enabling or affecting airbag 12 seatbelt pretensioner 14 deployment, control other vehicle features such as entertainment system 14, HVAC system controls 13 enable other controls 15, affect other vehicle features, or vehicle 10 may be prevented from being operated.

In block 302, a determination may be made that vehicle 10 is moving. If vehicle 10 is moving, a determination is made, in block 304, whether an occupant is present in, for example, second vehicle seat 28. If no occupant is present, no action is taken. If, however an occupant is detected, an action may be taken in block 306. The action may include stopping vehicle 10, slowing vehicle 10, enabling or controlling airbag 12, seatbelt pretensioner 19 deployment and/or seat heater/ventilation system local HVAC climate control 13 limiting other controls 15 from younger occupants, (limiting power consumption) and/or issuing a seatbelt warning. In block 402, a determination is made whether vehicle 10 has come to a stop. If vehicle 10 has come to a stop, occupant detection system 84 may again determine whether an occupant is in, for example, second vehicle seat 28 in block 406. If no occupant is detected, no action is taken. If an occupant is detected, an action may be taken in block 408 including preventing vehicle 10 from starting, issuing a seat belt alert, or the like. In block 500, occupant detection system 84 may power down.

Occupant detection system 84 in accordance with exemplary embodiments, employs biometric sensors to detect a physiological characteristic of an occupant in a vehicle seat. An occupant sitting on, for example, vehicle seat 28 presents a stronger signal to an associated one of first and second sensors 87 and 90 than would a child located in a child safety seat. Accordingly, the exemplary embodiments may employ sensed signal strength magnitude threshold to discriminate between an occupant that is directly on, for example, vehicle seat 28 and a child spaced from, for example, seat base surface 74 by a child safety seat. Exemplary embodiments may also detect, via sensed signal strength magnitude an out-of-position occupant that would be physically displaced away from one or more of first, second and fourth sensors 87, 90, 95. In addition, an empty seat could produce either the same signal magnitude as a child on the child safety seat or a lower magnitude response. Thus, the exemplary embodiments may be configured to distinguish between up to six different states depending on sensor location and the use of distinct threshold values for each condition: a) occupant on the seat, b) child in a child safety seat, c) no occupant present, d.) out of position occupant, e) animals and/or f), objects or anything other than human objects on the seat.

If employed, non-biometric sensor 106 may be utilized to discriminate other living objects that may be resting on, for example, vehicle seat 28. In an exemplary aspect, non-biometric sensor 106 may be configured to detect weight and controller 118 may determine whether the living object was below a pre-defined mass-based threshold. If below the mass-based threshold, an inference could be made that the living object may be a pet and not a human. Thus, the signal 130 may not be sent for living objects that are below a certain mass based threshold.

A confined sensing zone is established at each vehicle seat. The confined sensing zone allows the occupant detection system to discriminate between an occupant sitting in the seat and a child that may be in a child safety seat. The occupant is given a warning to use a primary restraint. A warning is not issued if the system determines that a child safety seat occupies the seat. Supplemental restraints such as airbags, seatbelt pretensioners, or other vehicle features such as entertainment systems, HVAC systems controls and other controls may be enabled or disabled. For instance, the child safety seat would not possess a physiological characteristic and thus not be detected by the occupant detection system. The exemplary embodiments ensure that a seat belt warning is issued, supplemental restraints are appropriately controlled and/or other vehicle features and actions are only taken when an occupant is seated on the seat surface.

The terms “about” and “substantially” are intended to include the degree of error associated with measurement of the particular quantity based upon the equipment available at the time of filing the application. For example, “about” and “substantially” can include a range of ±8% or 5%, or 2% of a given value.

While the above disclosure has been described with reference to exemplary embodiments, it will be understood by those skilled in the art that various changes may be made and equivalents may be substituted for elements thereof without departing from its scope. In addition, many modifications may be made to adapt a particular situation or material to the teachings of the disclosure without departing from the essential scope thereof. Therefore, it is intended that the present disclosure not be limited to the particular embodiments disclosed, but will include all embodiments falling within the scope thereof.

What is claimed is:

1. An occupant detection system comprising:
a vehicle seat including a seat back and a seat base;
a biometric sensor arranged in at least one of the seat back and the seat base, the biometric sensor being configured to detect a physiological characteristic of a seat occupant; and
a controller operatively connected to the biometric sensor, the controller being configured to provide an output signal in response to a physiological characteristic detected signal from the biometric sensor.

2. The occupant detection system according to claim 1, wherein the biometric sensor comprises at least one of an electrocardiogram (EKG) sensor, an electroencephalogram (EEG) sensor, an electromyography (EMG) sensor, a piezo electric sensor, a neural activity sensor, and a heart activity sensor.

3. The occupant detection system according to claim 1, wherein the biometric sensor is arranged in the seat base.

4. The occupant detection system according to claim 1, wherein the biometric sensor is arranged in the seat back.

5. The occupant detection system according to claim 1, wherein the biometric sensor is arranged between the seat back and the seat base.

6. The occupant detection system according to claim 1, wherein the biometric sensor establishes a physiological characteristic detection zone at the vehicle seat.

7. The occupant detection system according to claim 1, further comprising: a non-biometric sensor configured to detect a mass of a seat occupant.

8. A vehicle comprising:
a body defining, at least in part, an occupant compartment;
at least one vehicle seat arranged in the occupant compartment, the at least one vehicle seat including a seat back and a seat base;
a biometric sensor arranged in at least one of the seat back and the seat base, the biometric sensor being configured to detect a physiological characteristic of a seat occupant; and
a controller operatively connected to the biometric sensor, the controller being configured to provide an output signal in response to a physiological characteristic detected signal from the biometric sensor.

9. The vehicle according to claim 8, wherein the biometric sensor comprises at least one of a an electrocardiogram (EKG) sensor, an electroencephalogram (EEG) sensor, an electromyography (EMG) sensor, a piezo electric sensor, a neural activity sensor, and a heart activity sensor.

10. The vehicle according to claim 8, wherein the biometric sensor is arranged in the seat base.

11. The vehicle according to claim 8, wherein the biometric sensor is arranged in the seat back.

12. The vehicle according to claim 8, wherein the biometric sensor is arranged between the seat back and the seat base.

13. The vehicle according to claim 8, wherein the biometric sensor establishes a physiological characteristic detection zone at the at least one vehicle seat.

14. The vehicle according to claim 8, further comprising: a non-biometric sensor configured to detect a mass of a seat occupant.

15. A method of detecting an occupant in a vehicle comprising:
sensing a physiological characteristic at a vehicle seat.

16. The method of claim 15, wherein sensing the physiological characteristic includes detecting at least one of an electrical activity of a brain, electrical activity of a heart, heart movement, and breathing activity.

17. The method of claim 15, further comprising: detecting an occupant restraint status of the vehicle seat.

18. The method of claim 17, further comprising: providing a signal based on a presence of the physiological characteristic and occupant restraint status.

19. The method of claim 18, wherein providing the signal includes at least one of limiting operation status of the vehicle and warning the occupant to buckle a seatbelt, controlling an airbag, a seatbelt pretensioner, an entertainment system, and an HVAC system.

20. The method of claim 15, further comprising: detecting at least one of a child in a child safety seat resting on the vehicle seat, an occupant on the vehicle seat, an out-of-position occupant on the vehicle seat, an animal on the vehicle seat, and an inanimate object on the vehicle seat based on the sensed physiological characteristic.
